(19) United States
(12) Patent Application Publication
(10) Pub. No.: US 2014/0326246 A1
(43) Pub. Date: Nov. 6, 2014

(54) CUSHION ASSEMBLY HAVING COMPRESSION DAMPENING PORTION

(71) Applicant: KONINKLIJKE PHILIPS N.V., EINDHOVEN (NL)

(72) Inventors: Lauren Patricia Chodkowski, Pittsburgh, PA (US); Peter Chi Fai Ho, Pittsburgh, PA (US)

(21) Appl. No.: 14/361,522

(22) PCT Filed: Nov. 27, 2012
(86) PCT No.: PCT/IB2012/056759

§ 371 Date: May 29, 2014

Related U.S. Application Data
(60) Provisional application No. 61/567,147, filed on Dec. 6, 2011.

(51) Int. Cl. A61M 16/06

(52) U.S. Cl. CPC A61M 16/0611
USPC 128/206.24

(57) ABSTRACT

A cushion assembly for use in a patient interface device includes a first end portion adapted to engage a user's face; a second end portion opposite the first end portion, the second end portion being adapted to be coupled to a mask shell; a cushion portion having a wall portion extending between the first end portion and the second end portion, the wall portion including a stiffened portion; and a dampening portion including a dampening material disposed about the second end portion and adapted to be disposed between the stiffened portion and the mask shell.

### CROSS-REFERENCE TO RELATED APPLICATIONS

This patent application claims the priority benefit under 35 U.S.C. §119(e) of U.S. Provisional Application No. 61/567,147 filed on Dec. 6, 2011, the contents of which are herein incorporated by reference.

### BACKGROUND OF THE INVENTION

1. Field of the Invention

The present invention generally relates to a cushion assembly for use on a patient interface device in a pressure support system that supplies a flow of gas to the airway of a patient, and, more particularly, to a cushion assembly that includes a compression dampening portion. The invention further relates to a patient interface device that includes such a cushion.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation (NIV). It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), chronic obstructive pulmonary disease (COPD), or congestive heart failure (CHF).

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient.

Typically, patient interface devices include a mask shell having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because such masks are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. It is also important that the interface device provide a tight enough seal against a patient's face without discomfort. A problem arises in that in order for the mask to maintain a seal without any undue gas leaks around the periphery of the mask, the mask may be compressed against the patient's face.

Conventional CPAP masks that seal by compression commonly cause uncomfortable pressure points and do not adjust well to different anatomical facial features. The conventional concept of a compression seal is to generally displace tissues on the patient's face in order to achieve a uniform seal. Tissue displacement results in pressure points, skin markings (i.e., red marks), indentations, and overall prolonged discomfort.

Other conventional CPAP masks, such as self-articulating masks, do not account for over-tightening. Conventional masks utilizing air filled bellows tend to completely collapse when over tightened, thus eliminating the self-adjustment function.

### SUMMARY OF THE INVENTION

Accordingly, it is an object of the present invention to provide improved cushion assemblies and patient interface devices utilizing such cushion assemblies that overcome deficiencies in the known art.

As one aspect of the invention a cushion assembly for use in a patient interface device is provided. The cushion assembly comprises a first end portion adapted to engage a user's face; a second end portion opposite the first end portion, the second end portion being adapted to be coupled to a mask shell; a cushion portion having a wall portion extending between the first end portion and the second end portion, the wall portion including a stiffened portion; and a dampening portion including a dampening material disposed about the second end portion and adapted to be disposed between the stiffened portion and the mask shell.

The cushion portion and the dampening portion may be formed as two separate components that are selectively sealingly coupled.

The stiffened portion may be formed from a different material than the wall portion and the stiffened portion may be one of: encapsulated in the wall portion, externally coupled to the wall portion, and internally coupled to the wall portion.

The dampening portion may comprise a ring-shaped member formed from a solid elastic material.

The ring shaped member may disposed about the cushion portion and the cushion portion may pass through the ring-shaped member.

The cushion portion may be disposed about, and generally surround, the ring-shaped member.

The cushion portion and the dampening portion may be integrally formed.

The wall portion may comprise an inner wall portion and the cushion assembly may further comprise an outer wall portion disposed about the inner wall portion, the outer wall portion having a first end portion adapted to engage a user's face and a second end portion opposite the first end portion, the second end portion being coupled to the inner wall portion at or about the second end portion.

The stiffened portion may comprise a generally stiff gel and the dampening portion may comprise a gel softer than the generally stiff gel.

As another aspect of the invention a patient interface device is provided. The patient interface device comprises a mask shell and a cushion assembly having a first end portion adapted to sealingly engage a user's face and a second end portion opposite the first end portion, the second end portion coupled to the mask shell. The cushion assembly comprises a cushion portion having a wall portion extending between the first end portion and the second end portion. The wall portion includes a stiffened portion. The cushion assembly further comprises a dampening portion including a dampening material disposed about the second end portion between the stiffened portion and the mask shell.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a front isometric view of an example embodiment of a patient interface device according to the principles of the present invention shown (schematically) connected to a gas flow/pressure generating system to form a patient interface system;

FIG. 2 is a rear isometric view of the patient interface device of FIG. 1;

FIG. 3 is a top isometric exploded view of the patient interface device of FIGS. 1 and 2;

FIG. 4 is a cross-sectional view of the cushion assembly of the patient interface device of FIGS. 1 and 2 taken along the horizontal plane 4-4 of FIG. 2;

FIG. 5 is a cross-sectional view of another example embodiment of a patient interface device in accordance with the principles of the present invention taken along a horizontal plane;

FIG. 6 is an elevational view of a dampening portion of the patient interface device of FIG. 5;

FIG. 7 is a cross-sectional view of yet another example embodiment of a patient interface device in accordance with the principles of the present invention taken along a horizontal plane;

FIG. 8 is an elevational patient side view of the cushion assembly of the patient interface device of FIG. 7;

FIGS. 9A-9D are elevational views of examples of different shaped dampening portions in accordance with the principles of the present invention;

FIGS. 10 and 11 are examples of the use of cut-outs and voids in a solid elastic material in accordance with the principles of the present invention to selectively vary the stiffness of the elastic material;

FIG. 12 is cross-sectional views of portions of three example dampening portions in accordance with the principles of the present invention; and

FIG. 13 is a cross-sectional view of yet another example embodiment of a patient interface device in accordance with the principles of the present invention taken along a horizontal plane.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality) and the singular form of “a”, “an”, and “the” include plural referents unless the context clearly indicates otherwise.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

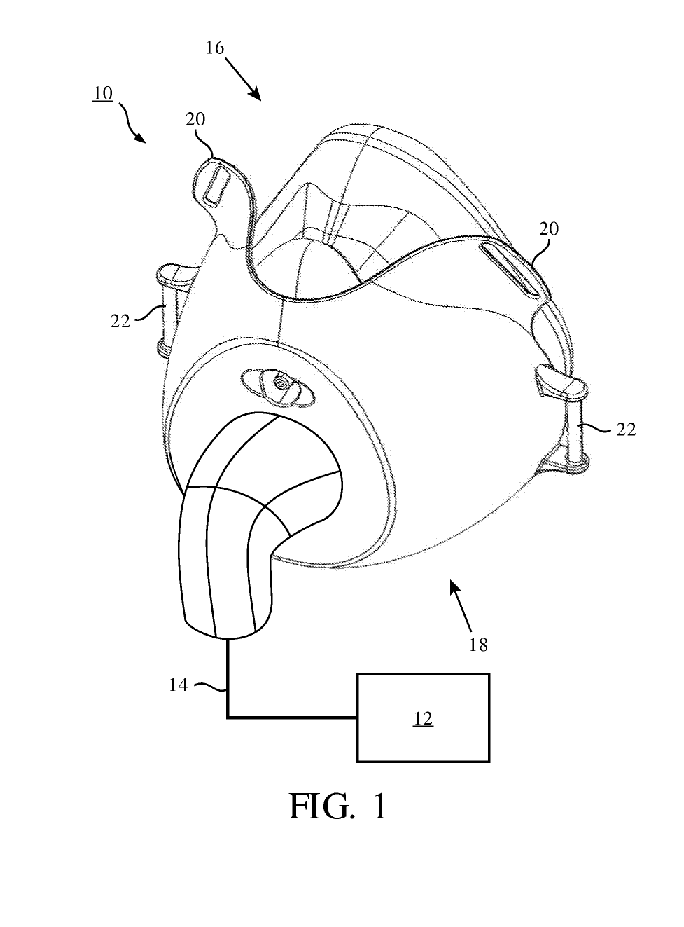

FIGS. 1-4 illustrate an exemplary embodiment of a patient interface device 10 and components thereof according to the principles of the present invention. Patient interface device 10 communicates a flow of breathing gas between the patient's airway and a pressure/flow generating system 12 (shown schematically), such as a ventilator, CPAP device, or variable pressure device, e.g., a BiPAP® device manufactured and distributed by Philips Respironics, Inc. of Pittsburgh, Pa., or an auto-titration pressure support system.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating system 12 is also referred to as a gas flow generating device, because flow results when a pressure gradient is generated. The present invention contemplates that pressure/flow generating system 12 is any conventional system for delivering a flow of gas to an airway of a patient or for elevating a pressure of gas at an airway of the patient, including the pressure support systems summarized above and non-invasive ventilation systems.

Communicating a flow of breathing gas between the patient's airway and pressure/flow generating system 12 includes delivering a flow of breathing gas to the patient from the pressure/flow generating device and exhausting a flow of gas from the patient to ambient atmosphere. The system for delivering a breathing gas to a patient according to the present invention comprises pressure/flow generating system 12 that produces a flow of gas, and a conduit 14, which is also referred to as a patient circuit, having a first end portion (not numbered) operatively coupled to the gas flow generating device and a second end portion (not numbered). Conduit 14 carries the flow of gas from pressure/flow generating device 12 during operation of the system to patient interface device 10, which is coupled to the second end portion of conduit 14. Conduit 14 corresponds to any conduit suitable for communicating the flow of gas form the pressure/flow generating system to the patient interface device. An example of a typical conduit is a flexible tube. A headgear assembly, which is not shown in the figures, attaches patient interface device 10 to the patient's head.

Patient interface device 10 includes a cushion assembly, generally indicated at 16, and a mask shell 18 having a patient side and opposite thereto, an outer side. Attached to outer side of mask shell 18 is a conduit coupling member (not numbered) that couples mask shell 18 to conduit 14 so that a flow of gas is communicated to the interior of the patient interface device for subsequent delivery to the patient. Conversely, gas from the patient is communicated from the patient interface device into conduit 14, where an exhaust port is located. Mask shell 18 is preferably a generally rigid shell, and, in an exemplary embodiment of the present invention is formed from rigid plastic, such as polycarbonate. It is to be understood that the present invention contemplates that one or more of the size, shape, or composition of mask shell 18 may be varied without varying from the scope of the present invention.

In the illustrated embodiment of FIG. 1, mask shell 18 has a generally rounded triangular shape and is provided with upper and lower headgear attaching elements 20, 22, which cooperate with corresponding attachment elements on headgear straps (not illustrated) for securely mounting patient interface device 10 on the head of a user. It is to be understood that the present invention contemplates using any conventional connection assembly to attach a headgear or headgear strap to mask shell 18 or other suitable shell arrangement. It is to be further understood that the present invention also contemplates that mask shell 18 may further include a forehead support portion having headgear attaching elements for connection to further headgear straps. The present invention also contemplates providing a post or other protrusion at the upper portion of the shell, i.e., the portion overlying the bridge of the nose, to which the headgear can be attached.

The present invention contemplates that the headgear suitable for use with patient interface device 10 is any conventional headgear used in the patient interface field. For example, without limitation, a typical headgear assembly comprises a headpiece that overlies a portion of the patient's crania and with headgear straps extending therefrom to adjustably connect the headgear to the mask.

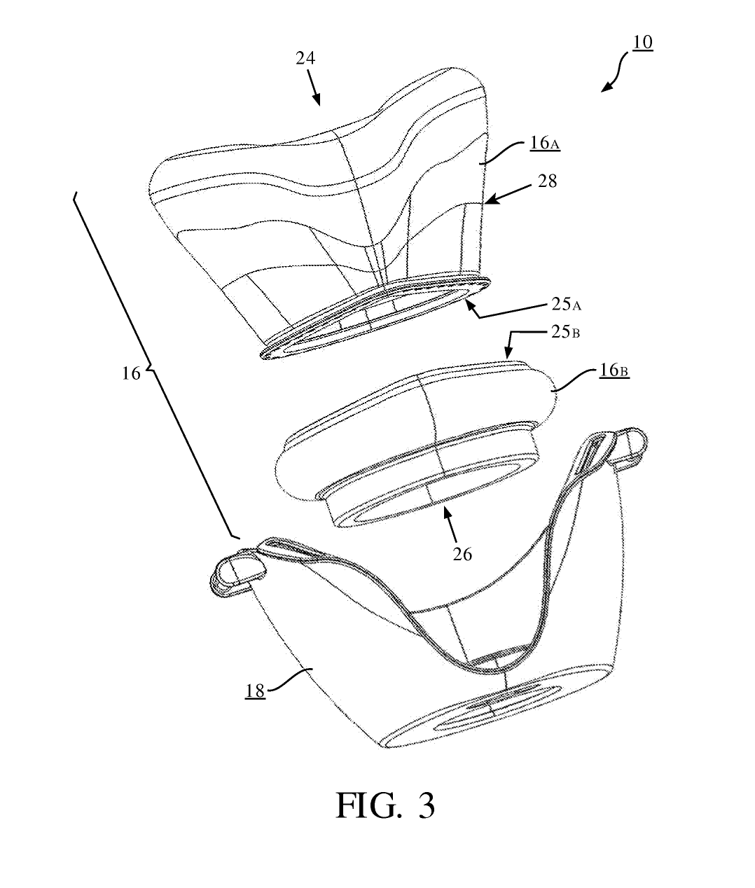

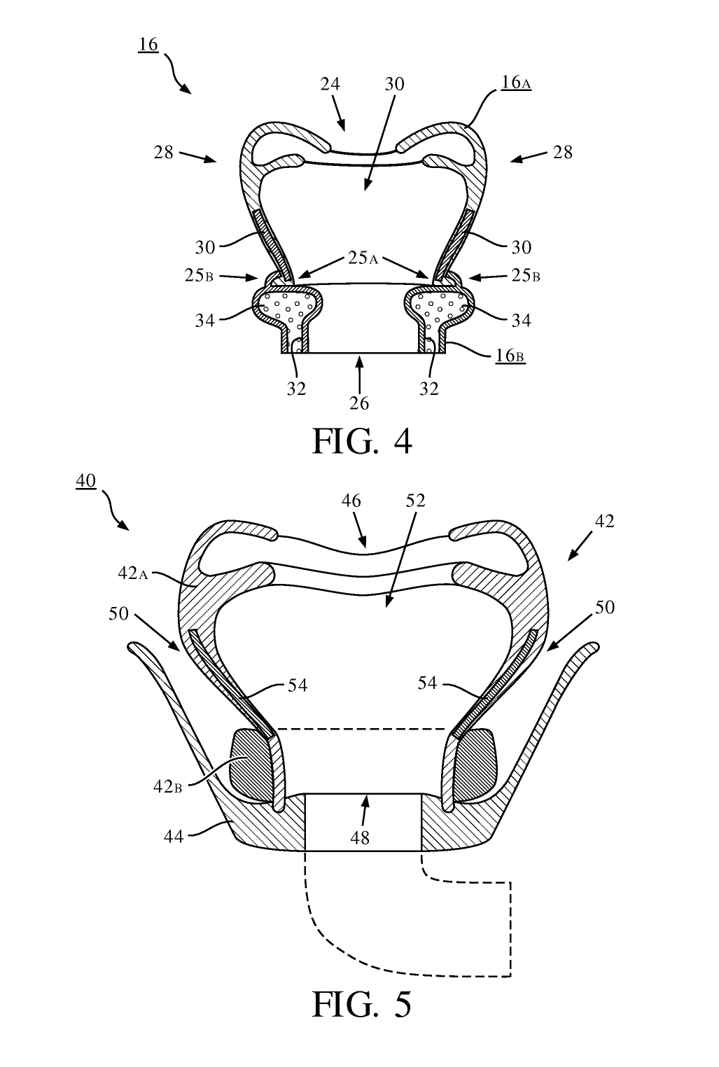

Referring to FIGS. 3 and 4, cushion assembly 16 includes a cushion portion 16a and a dampening portion 16b. In the embodiment illustrated in FIGS. 1-4, cushion portion 16a and dampening portion 16b are formed as separate members that cooperatively engage and interlock together to form cushion assembly 16. However, as discussed further below, it is to be understood that such cushion and dampening portions may be formed as integral portions of a single cushion. In an exemplary embodiment, cushion portion 16a is formed of a soft, cushiony, elastomeric material, such as silicone, appropriately soft thermoplastic elastomers, closed cell foam, thin materials, silicone, or any other material or combination of suitable materials.

Cushion assembly 16 includes a first end portion 24 adapted to engage, preferably in a sealing manner, an inner perimeter of the face of a user and a second end portion 26 generally opposite first end portion 24 that is adapted to be coupled to a mask (such as mask shell 18 in FIGS. 1-3). In the embodiment illustrated in FIGS. 1-4, cushion portion 16a extends from first end portion 24 to a connecting portion 25a and dampening portion 16b extends from another connecting portion 25b to second end portion 26. As shown in FIGS. 3 and 4, connecting portions 25a and 25b are sized and configured to cooperatively engage and interlock with each other in such a manner that cushion portion 16a and dampening portion 16b may be selectively sealingly coupled to each other.

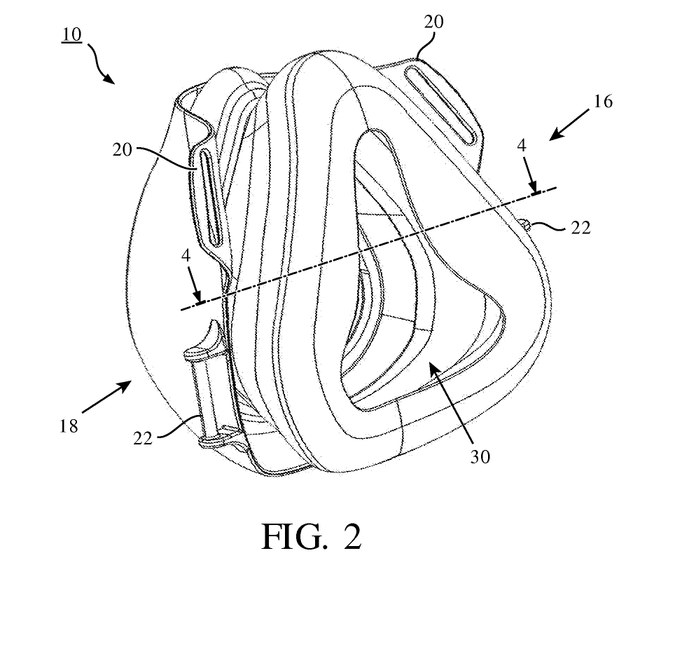

Cushion portion 16a includes a wall portion 28 which extends between first end portion 24 and coupling portion 25a. A nose receiving cavity 30 (FIGS. 2 and 4) adapted to receive at least a portion of a user's nose is defined in the interior of cushion assembly 16 by wall portion 28. When cushion portion 16a and dampening portion 16b are interlocked, such as shown in the cross-sectional view of FIG. 4, wall portion 28 extends generally between first end portion 24 and second end portion 26 of cushion assembly 16.

As shown in the cross-sectional view of FIG. 4, wall portion 28 includes a rigid or stiffened portion 30 (as compared to the adjacent material of wall portion 28) positioned and adapted to generally prevent cushion portion 16a from collapsing when patient interface device 10 is mounted, and subsequently tightly strapped to the face of a patient. In an exemplary embodiment, stiffened portion 30 is formed from a rigid plastic or other suitable rigid or semi-rigid material relative to the material from which the remainder of cushion portion 16a is formed. As shown in the example embodiment of FIG. 4, stiffened portion 30 may be integrally formed therewith (and encapsulated therein) wall portion 28. However, it is to be understood that the present invention contemplates that stiffened portion 30 may also be coupled to an outer or inner portion (not numbered) or other suitable portion of wall portion 28.

Continuing to refer to the cross-sectional view of FIG. 4, dampening portion 16b includes a compartment or bladder 32 formed with a gel or other suitable dampening material 34 disposed therein. Dampening material 34 is positioned generally between stiffened portion 30 and mask shell 18 such that dampening material 34 acts to dampen and evenly disperse forces resulting from tightening of mask shell 18 (via headgear straps) on the head of a patient which would otherwise typically be absorbed directly by concentrated regions of facial tissue at or about first end portion 24 of cushion assembly 16. As mask shell 18 is mounted and tightened to a patient's face, dampening portion 16b compresses and articulates to distribute forces at first end portion 24 of cushion assembly 16 evenly across a variety of facial geometries.

Similar to the cross-sectional view of FIG. 4, FIG. 5 shows a horizontal cross-sectional view of another embodiment of a patient interface device 40 according to the principles of the present invention. Like patient interface device 10 previously discussed, patient interface device 40 includes a cushion assembly, generally indicated at 42 and a mask shell 44 having a patient side and opposite thereto, an outer side. Similar to cushion assembly 16 previously discussed, cushion assembly 42 includes a cushion portion 42a and a dampening portion 42b formed as separate members.

In an exemplary embodiment, cushion portion 42a is formed of a soft, cushiony, elastomeric material, such as silicone, appropriately soft thermoplastic elastomers, closed cell foam, thin materials, or any combination of suitable materials. Cushion portion 42a includes a first end portion 46 adapted to engage an inner perimeter of the face of a user to form a seal therewith, and a second end portion 48 generally opposite first end portion 46 that is adapted to be coupled to a mask (such as mask shell 44) through any suitable mechanism. Cushion portion 42a includes a wall portion 50 which extends between first end portion 46 and second end portion 48 and defines a nose receiving cavity 52 adapted to receive at least a portion of a user's nose therein.

Wall portion 50 includes a rigid or stiffened portion 54 positioned and adapted to generally prevent cushion portion 42a from collapsing when patient interface device 40 is mounted, and subsequently tightly strapped to the face of a patient. In an exemplary embodiment, stiffened portion 54 is formed from a rigid plastic or other suitable rigid or semi-rigid material and may be formed integrally with wall portion 50 (as shown in FIG. 5) or may be coupled to an outer or inner portion (not numbered) or other suitable portion of wall portion 50.

Unlike dampening portion 16b (previously described) which utilizes dampening material 34 disposed in a bladder 32 formed therein, dampening portion 42b is formed from a solid elastic material which does not require a housing to maintain a desired shape. As shown in the elevational view of FIG. 6, dampening portion 42b is formed generally as a donut-shaped ring member. As shown in the cross-sectional view of FIG. 5, dampening portion 42b is disposed about, and generally surrounds, second end portion 48 of cushion portion 42a between stiffened portion 54 and mask shell 44 such that cushion portion 42a passes through dampening portion 42b. Similar to dampening portion 16b previously discussed, dampening portion 42b acts to dampen and evenly disperse forces resulting from tightening of mask shell 44 (via headgear straps, not shown) on the head of a patient which would otherwise typically be absorbed directly by concentrated regions of facial tissue. As mask shell 44 is mounted and tightened to a patient's face, dampening portion 42b deforms and articulates to distribute forces more evenly across a variety of facial geometries.

It is to be understood that dampening portion 42b does not fold or inflate to achieve articulation, but instead utilizes the compression and/or deformation properties of different solid elastic materials to allow articulation even as the mask shell 44, and thus patient interface device 40, is tightened to the face of a patient. By evening the distribution of forces, pressure points are greatly reduced or eliminated, thus minimizing red marks and indentations and improving seal and overall comfort of patient interface device 40.

FIG. 7 shows a cross-sectional view of another embodiment of a patient interface device 60 having a mask shell 62 and a cushion assembly 64 according to the principles of the present invention. Interface device 60 is generally constructed and functions in a similar manner to interface device 40, however, cushion assembly 64 utilizes a dampening portion 64b, formed from a solid elastic material, disposed inside a cushion portion 64a between stiffened portions 66 and mask shell 62 such that cushion portion 64a is disposed about, and generally surrounds, dampening portion 64b. FIG. 8 shows an elevational patient side view of cushion assembly 64 and the general positioning of dampening portion 64b (shown in hidden line) relative to cushion portion 64a.

It is to be understood that the present invention contemplates that gel filled bodies may be employed in place of solid elastic materials and vice versa in dampening portions depending on the particular needs of a specific application. It is also to be understood that the present invention contemplates that the shape (as viewed from a patient or mask shell side) of the dampening material used in a particular dampening portion may be varied depending on the application and how the forces from a mask shell need to be disbursed.

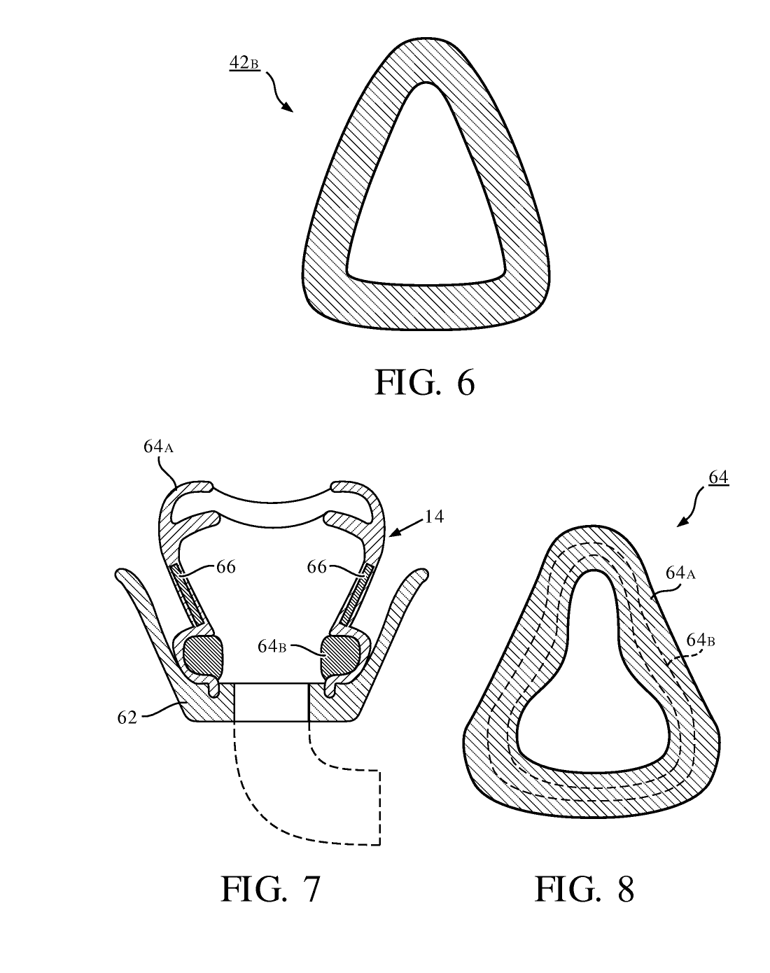

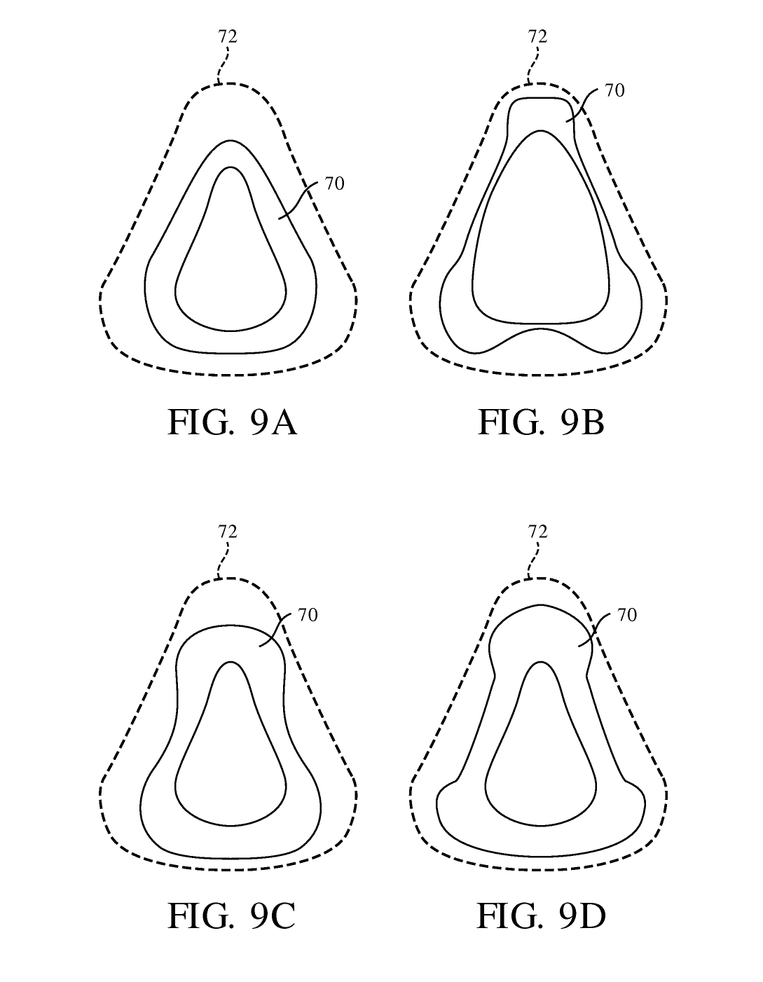

FIGS. 9A-9D, respectively, show elevational views of example embodiments of dampening portions 70 of differing shape compared to an example cushion portion 72 (shown in hidden line) in accordance with the principles of the present invention. It is to be understood that the present invention contemplates that such dampening portions 70 may be formed from solid elastic materials having suitable dampening properties or from flexible bladder structures having a suitable gel material or materials disposed therein.

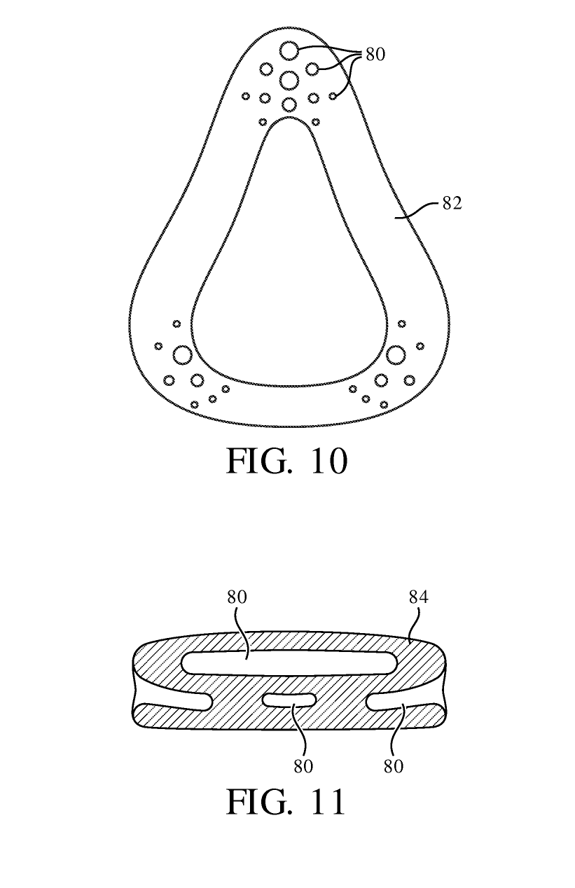

FIGS. 10 and 11, respectively, show elevational and cross-sectional views of dampening portions formed from solid elastic materials 82, 84 having cut-outs and voids 80 of predetermined shape and size in order to reduce the stiffness of selected portions of the dampening portions.

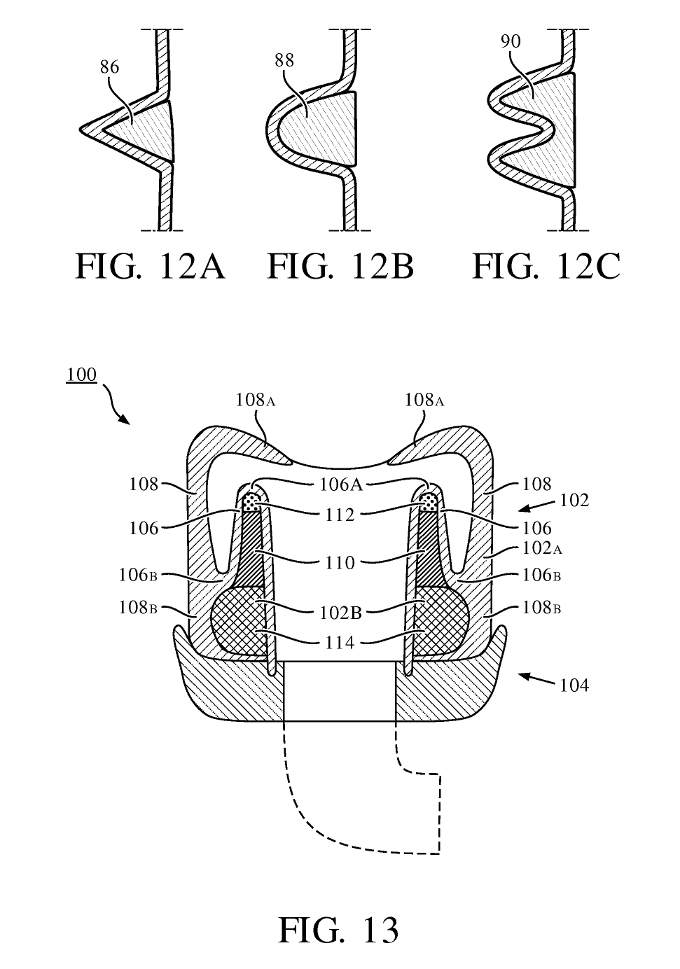

FIG. 12 shows cross-sectional views of portions of three example dampening portions 86, 88 and 90. Dampening portion 86 has a generally triangular-shaped profile, dampening portion 88 has a somewhat rounded generally trapezoidal-shaped profile, and dampening portion 90 has a stacked double triangular shaped profile. By varying the cross-sectional profile of the dampening portions, the dampening properties of such portions may be varied in order to produce desired dampening characteristics for a specific application.

Similar to the cross-sectional views of FIGS. 4, 5 and 7, FIG. 13 shows a horizontal cross-sectional view of another example embodiment of a patient interface device 100 according to the principles of the present invention. Like patient interface devices 10, 40 and 60 previously discussed, patient interface device 100 includes a cushion assembly, generally indicated at 102 and a mask shell 104 having a patient side and opposite thereto, an outer side. Similar to cushion assemblies 16, 42 and 64 previously discussed, cushion assembly 102 includes a cushion portion 102a and a dampening portion 102b.

Unlike the embodiments previously discussed, cushion portion 102a includes an inner wall portion 106 and an outer wall portion 108, disposed generally about inner wall portion 106. In an exemplary embodiment, outer wall portion 108 is formed of a soft, cushiony, elastomeric material, such as silicone, appropriately soft thermoplastic elastomers, closed cell foam, thin materials, or any combination of suitable materials and includes a first end portion 108a adapted to engage, preferably in a sealing manner, an inner perimeter of the face of a user and a second end portion 108b generally opposite first end portion 108a. Inner wall portion 106 includes a first end portion 106a adapted to directly engage an further inner perimeter of the face of a user or, as shown in FIG. 13, indirectly engage the face of a user via an inner portion (not numbered) of first end portion 108a that is engaged with the face of a user. Inner wall portion 106 further includes a second end portion 106b disposed generally opposite first end portion 106a that is coupled to, or integrally formed with, second end portion 108b of outer wall portion 108.

Inner wall portion 106 includes a first portion 110 having a generally stiff gel or other suitable rigid or semi-rigid material disposed therein. Inner wall portion 106 further includes a second portion 112 disposed generally at or about first end 106a having a generally soft gel or other suitable material disposed therein and a third portion 114 disposed at or about second end 106b having a semi-soft gel or other suitable dampening material disposed therein. Similar to the embodiments previously discussed, third portion 114 acts as a dampening portion disposed generally between generally stiff first portion 110 and mask shell 104 which acts to distribute forces of first end 106a of inner wall portion 106 more evenly across a variety of facial geometries.

It is to be appreciated that the present invention is not intended to be limited to the mask or cushion shapes described herein but instead may be employed with masks and cushion of various other shapes or designs as long as the dampening portion is disposed generally between a stiffened portion of the cushion wall and the mask.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A cushion assembly for use in a patient interface device, the cushion assembly comprising:
a first end portion adapted to engage a user's face;
a second end portion opposite the first end portion, the second end portion being adapted to be coupled to a mask shell;
a cushion portion having a wall portion extending between the first end portion and the second end portion, the wall portion including a stiffened portion including one or more of: a generally stiff gel, a rigid material or a semi-rigid material; and
a dampening portion including a dampening material softer than the stiffened portion, the dampening portion disposed about the second end portion and adapted to be disposed between the stiffened portion and the mask shell.

2. The cushion assembly of claim 1, wherein the cushion portion and the dampening portion are formed as two separate components that are selectively sealingly coupled.

3. The cushion assembly of claim 1, wherein the stiffened portion is formed from a different material than the wall portion and wherein the stiffened portion is one of: encapsulated in the wall portion, externally coupled to the wall portion, and internally coupled to the wall portion.

4. The cushion assembly of claim 1, wherein the dampening portion comprises a ring-shaped member formed from a solid elastic material.

5. The cushion assembly of claim 4, wherein the ring shaped member is disposed about the cushion portion and the cushion portion passes through the ring-shaped member.

6. The cushion assembly of claim 4, wherein the cushion portion is disposed about, and generally surrounds, the ring-shaped member.

7. The cushion assembly of claim 1, wherein the cushion portion and the dampening portion are integrally formed.

8. The cushion assembly of claim 1, wherein the wall portion comprises an inner wall portion and wherein the cushion assembly further comprises an outer wall portion disposed about the inner wall portion, the outer wall portion having a first end portion adapted to sealingly engage a user's face and a second end portion opposite the first end portion, the second end portion being coupled to the inner wall portion at or about the second end portion.

9. (canceled)

10. A patient interface device comprising:
a mask shell; and
a cushion assembly having a first end portion adapted to sealingly engage a user's face and a second end portion opposite the first end portion, the second end portion coupled to the mask shell, the cushion assembly comprising:
a cushion portion having a wall portion extending between the first end portion and the second end portion, the wall portion including a stiffened portion including one or more of: a generally stiff gel, a rigid material or a semi-rigid material; and
a dampening portion including a dampening material softer than the stiffened portion, the dampening portion disposed about the second end portion between the stiffened portion and the mask shell.

11. The patient interface device of claim 10, wherein the cushion portion and the dampening portion are integrally formed.

12. The patient interface device of claim 10, wherein the cushion portion and the dampening portion are formed as two separate components that are selectively sealingly coupled.

13. The patient interface device of claim 10, wherein the stiffened portion is formed from a different material than the wall portion and wherein the stiffened portion is one of: encapsulated in the wall portion, externally coupled to the wall portion, and internally coupled to the wall portion.

14. The patient interface device of claim 10, wherein the dampening portion comprises a ring-shaped member formed from a solid elastic material.

15. The patient interface device of claim 14, wherein the ring shaped member is disposed about the cushion portion and the cushion portion passes through the ring-shaped member.

16. The patient interface device of claim 14, wherein the cushion portion is disposed about, and generally surrounds, the ring-shaped member.

17. (canceled)

18. The patient interface device of claim 10, wherein the wall portion comprises an inner wall portion and wherein the cushion assembly further comprises an outer wall portion disposed about the inner wall portion, the outer wall portion having a first end portion adapted to sealingly engage a user's face and a second end portion opposite the first end portion, the second end portion being coupled to the inner wall portion at or about the second end portion.

19. (canceled)
